(19) United States
(12) Patent Application Publication
(10) Pub. No.: US 2010/0087742 A1
(43) Pub. Date: Apr. 8, 2010

(54) PHYSIOLOGICAL CHARACTERISTIC DETERMINATION FOR A MEDICAL DEVICE USER

(75) Inventors: Corey J. Bishop, Midvale, UT (US); Nathanael O. Mason, Salt Lake City, UT (US); Robert L. Lux, Park City, UT (US)

Correspondence Address: AUSTIN RAPP & HARDMAN, 170 South Main Street, Suite 735, SALT LAKE CITY, UT, 84101, US

(73) Assignee: IHC Intellectual Asset Management, LLC, Salt Lake City, UT (US)

(21) Appl. No.: 12/570,944

(22) Filed: Sep. 30, 2009

Related U.S. Application Data
(60) Provisional application No. 61/101,624, filed on Sep. 30, 2008.
(60) Provisional application No. 61/182,639, filed on May 29, 2009.

(51) Int. Cl. A61B 5/02; A61M 1/10

(52) U.S. Cl. 600/481; 600/16

(57) ABSTRACT

A method for determining a physiological characteristic of a patient on which a medical device is applied is disclosed. Electric current data is obtained from the medical device. The electric current data is filtered to produce filtered data. The physiological characteristic is determined based on the filtered data.

### RELATED APPLICATIONS

This application is related to and claims priority from U.S. Provisional Patent Application Ser. No. 61/101,624 filed Sep. 30, 2008 for “Systems and Methods for Determining Whether the Aortic Valve Is Opening and Further Investigation of Calculating Flow and Ejection Fraction,” and from U.S. Provisional Patent Application Ser. No. 61/182,639 filed May 29, 2009 for “Systems and Methods for Determining Whether an Aortic Valve Is Opening in Left Ventricular Assist Device Patients,” both of which are incorporated herein by reference.

### TECHNICAL FIELD

The present invention relates generally to medical devices. More specifically, the present invention relates to systems and methods for determining a physiological characteristic of a patient on which a medical device is applied.

### BACKGROUND

Heart failure is one of the world's leading causes of death, affecting more than 4.5 million people in the U.S. The prevalence of heart failure is expected to increase 10 to 15% by the year 2020. Patients' heart failure conditions, in severe cases, cannot be medically managed and their only option is to receive a heart transplant; however, there is a tremendous shortage of transplantable hearts worldwide. Patients now have an additional option of receiving a Left Ventricular Assist Device (LVAD) which is used for bridging patients to heart transplants, bridge to recovery or as Destination Therapy (implanting the LVAD indefinitely).

The LVAD decreases the work load of the left ventricle of the heart by producing both pressure and volume unloading of the heart. First generation LVADs were made to pump blood in a pulsatile manner because it was believed by the manufacturing companies that pulsatility was optimal for the circulatory system. These pulsatile LVADs have bearings and moving parts which limit the durability and life of the pump. To overcome durability issues, LVADs with less moving parts were designed. The life of these continuous flow pumps is now estimated to be six to eight years. Therefore, there is a need for improved systems and methods related to the operation of LVADs.

### BRIEF DESCRIPTION OF THE DRAWINGS

FIG. 1 is a block diagram illustrating a system for determining whether an aortic valve is opening in a patient with a left ventricular assist device (LVAD);

FIG. 2 is a flow diagram illustrating a method for determining whether an aortic valve is opening in an LVAD patient;

FIG. 3 is a block diagram illustrating a system for determining whether an aortic valve (AV) is opening in an LVAD patient;

FIG. 4 is a flow diagram illustrating a method for preparing data before determining whether an aortic valve is opening in an LVAD patient;

FIG. 5 is a flow diagram illustrating a method for filtering and organizing data before determining whether an aortic valve is opening in an LVAD patient;

FIG. 6 is a waveform illustrating calibrated data;

FIG. 7 is a waveform illustrating overlapped data;

FIG. 8 is a flow diagram illustrating a method for Principal Component Analysis (PCA);

FIG. 9 is a waveform illustrating eigenvectors;

FIG. 10 is a waveform illustrating eigenvalues;

FIG. 11 is a waveform illustrating the projections of data onto the first eigenvector;

FIG. 12 is a screenshot illustrating one possible configuration of an LVAD aortic valve opening analyzer clinical application; and

FIG. 13 is a block diagram illustrating various components that may be utilized in a computing device in a system for determining whether an AV is opening in LVAD patients.

### DETAILED DESCRIPTION

A method for determining a physiological characteristic of a patient on which a medical device is applied is disclosed. Electric current data is obtained from the medical device. The electric current data is filtered to produce filtered data. The physiological characteristic is determined based on the filtered data.

The medical device may be a left ventricular assist device (LVAD) and the physiological characteristic may be whether an aortic valve in the patient is opening. Alternatively, the physiological characteristic may be a blood pressure of the patient. The rotations per minute (RPM) of a pump in the LVAD may be adjusted based on the determination of the physiological characteristic. A rate of blood flow through the LVAD may be determined by integrating an area under a curve defined by the electric current data.

The contractility of the patient's heart may be determined using a magnitude of the electric current data that is proportional to contractility when volume status is normalized. Whether to remove the LVAD from the patient may be determined based on the contractility determination.

In one configuration, the physiological characteristic may be determined using Principal Component Analysis. Furthermore, in Principal Component Analysis, a covariance matrix may be determined based on the filtered data. Eigenvalues and eigenvectors may be calculated based on the covariance matrix. The eigenvectors may be projected onto the filtered data, and the physiological characteristic may be determined based on a characteristic of the projection.

An apparatus for determining a physiological characteristic of a patient on which a medical device is applied is also disclosed. The apparatus includes a processor and memory in electronic communication with the processor. Executable instructions are stored in the memory. The instructions are executable to obtain electric current data from the medical device. The instructions are also executable to filter the electric current data to produce filtered data. The instructions are also executable to determine the physiological characteristic based on the filtered data.

A computer readable medium that includes executable instructions is also disclosed. The instructions are executable for obtaining electric current data from a medical device applied to a patient. The instructions are also executable for filtering the electric current data to produce filtered data. The instructions are also executable for determining a physiological characteristic of the patient based on the filtered data.

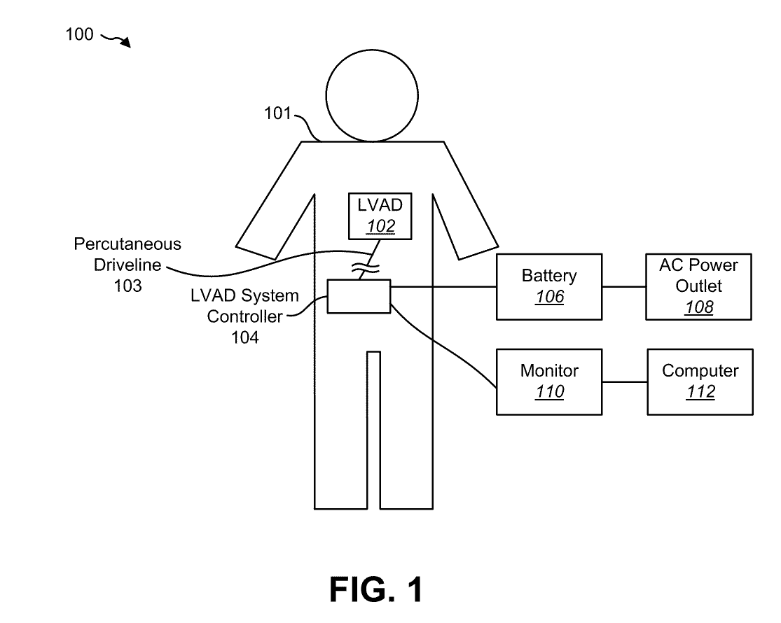

FIG. 1 is a block diagram illustrating a system 100 for determining whether an aortic valve is opening in a patient 101 with a left ventricular assist device (LVAD) 102. The LVAD 102 may direct blood from the left ventricle of the patient's 101 heart and into the aorta bypassing the aortic valve (AV). As used herein, the term “LVAD” refers to any rotary, continuous flow, mechanical device that is used to partially or completely replace the function of the heart. For example, the HeartMate II LVAD by Thoratec Corporation is an example of an LVAD 102 that may be used with the present systems and methods. The patient 101 may require an LVAD 102 for a variety of reasons, e.g., recovery from a heart attack or heart surgery, as a bridge to a heart transplant, or for congestive heart failure.

The LVAD 102 may be connected to an LVAD system controller 104 via a percutaneous driveline 103 that tunnels through the abdomen of the patient 101. Alternatively, the LVAD 102 may communicate with the LVAD system controller 104 via a wireless link. The LVAD system controller 104 controls the rotations per minute (RPM) of the propulsion mechanism in the LVAD 102, monitors operation of the LVAD 102, and provides alerts if the LVAD 102 malfunctions. The system 100 may further include one or more batteries 106 that provide power to the LVAD 102 and LVAD system controller 104. The battery 106 may be portable and may be rechargeable using an alternating current (AC) power outlet 108. The system 100 may also include an external monitor 110 that communicates with the LVAD system controller 104 and displays information from the LVAD 102. Furthermore, this information may also be processed and/or displayed by a computer 112, i.e., any device capable of displaying and/or transforming data.

When a non-pulsatile LVAD 102 is running too fast (i.e., the RPM of the LVAD 102 is too high), all of the blood that enters the left ventricle of a heart may exit through an inflow conduit of the LVAD 102. Consequently, no blood volume may flow through its native route, i.e., the AV. When not enough blood volume flows through the AV, eddies may form in ways that promote thrombogenesis, i.e., clot formation. Thrombogenesis may result in neurological events, such as transient ischemic attacks and cerebrovascular accidents, which may place the patient 101 at a greater health risk. In addition, when the AV is not opening, the AV is more likely to undergo stenosis and fusion, which further promotes disturbances in blood flow and thrombogenesis.

Achieving the optimal degree of LVAD 102 mechanical circulatory support for the LVAD' s 102 RPM may be difficult. In other words, it may be desirable that the RPM of the LVAD 102 be low enough that the AV is opening but high enough that the patient 101 receives adequate systemic circulation. One possible method of determining this balance between mechanical circulatory support and RPM of the LVAD 102 may utilize echocardiograms taken at regular intervals post-LVAD-implantation, e.g., every few months. It may be inconvenient and unnecessarily expensive to require regular echocardiograms for the lifetime of the LVAD 102, which may be indefinite. Another possible method is to measure the systolic and diastolic pressure differential and assume opening if the differential is greater than a given amount, but this may be inaccurate and lead to general assumptions of the entire population and thus is not patient specific. Instead, the present system 100 describes a novel approach to determine whether the AV is opening by analyzing the LVAD's 102 electric current. This AV detection algorithm could potentially be incorporated into the LVAD system controller 104 to continuously monitor and regulate the RPM of the LVAD 102 for determining the optimal medium between providing systemic circulation support and mal-effects associated with aortic valve akinesis. Adaptable continuous control of rotary LVADs 102 may decrease neurological events and AV stenosis or fusion for the patient 101. The system 100 may use signal processing techniques to analyze the electric current used by the LVAD 102 to determine when the AV is opening and when the AV is continuously closed.

In one configuration, the system 100 may use a modified Kahrunen-Loève Transform Analysis. The Kahrunen-Loève Transformation is more commonly known as Principal Component Analysis (PCA). PCA may be used to characterize the trends between the AV opening and the electric current usage of the LVAD 102. PCA may be used to describe a dataset using a subset of linear combinations, known as eigenvectors. PCA may aid visual examination and interpretation of complex data through data reduction and structure detection. Eigenvectors are indicators of shared signal behavior. Eigenvectors' associated eigenvalues may allow the system 100 to rank the order of contributing importance to the overall electrical signal. Through echocardiogram comparison it is possible to determine which eigenvectors, if any, reveal behavior that consistently determines whether the AV is opening. With PCA, the appropriate LVAD 102 RPM may then be determined for a desired AV opening ratio without continuous echocardiograms. The optimal AV opening ratio may be any suitable number. For example, it may be desirable for the AV to open at least once every three beats. The present systems and methods describe the structural changes in the electric current of the LVAD 102 when the AV is opening and when the AV is continually closed. Based on these findings, a user-friendly waveform viewer for continuous flow, non-pulsatile LVADs 102 may be used in a clinical setting.

Although described using an LVAD 102 to determine whether an AV is opening, the present systems and methods may be used with any suitable medical device to determine other physiological characteristics in a patient. For example, a controller may determine whether a pulmonary valve is opening in a right ventricular assist device (RVAD) patient. The present systems and methods may also be used with urinary tube data. Alternatively, a relative blood pressure of a patient may be obtained by correlating the amplitude of the electrical sinusoidal signal with some proportionality constant and normalizing the power consumption of the ventricular assist device (VAD), although this relationship may not be linear. In other words, this may not result in an actual systolic value and diastolic value, (i.e., 120 mm Hg/80 mm Hg), but rather a relative value for systole and diastole, (i.e., 120-80 mm Hg=40 mm Hg), which is the pulsatile pressure often used by physicians.

In one configuration, the present systems and methods may also be used with a pulsatile VAD. However, pulsatile VADs may beat asynchronously with the native heart, so the signal for the AV may be in different locations. In this configuration, the signal from the VAD may be filtered and only the heart contributory frequencies due to the addition of pulsatile flow velocities may be used.

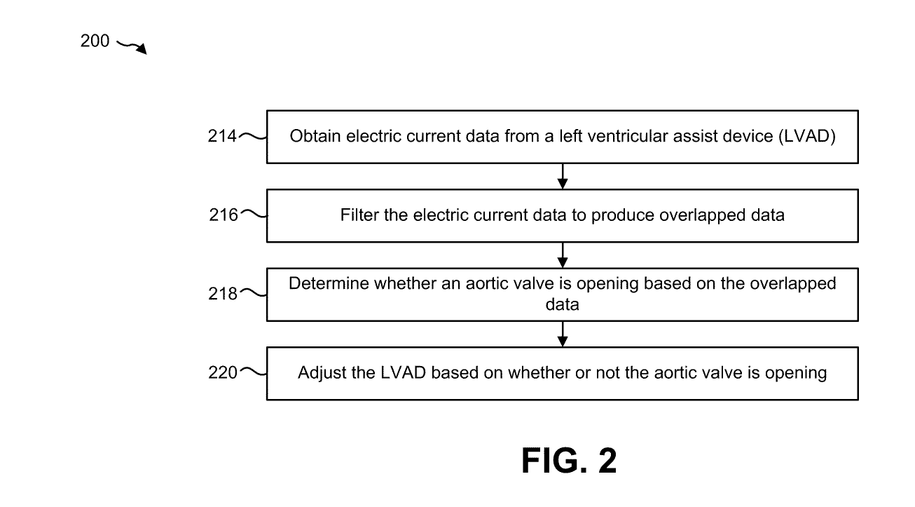

FIG. 2 is a flow diagram illustrating a method 200 for determining whether an aortic valve is opening in an LVAD 102 patient 101. The method 200 may be performed by an LVAD system controller 104 and/or a monitor 110 and computer 112. The LVAD system controller 104 may obtain 214 electric current data from an LVAD 102. This may include converting data from a proprietary format to a usable format. The LVAD system controller 104 may also filter 216 the electric current data to produce overlapped data. This may include using various signal processing techniques, e.g., Fast Fourier Transform (FFT), peak detection, etc. The LVAD system controller 104 may also determine 218 whether the AV is opening based on the overlapped data. This may also include using signal processing techniques, e.g., FFT, PCA, etc. The LVAD system controller 104 may also adjust 220 the LVAD 102 based on whether or not the aortic valve is opening. For example, if the aortic valve is not opening, the LVAD system controller 104 may reduce the RPM of the LVAD 102 so that the flow through the LVAD 102 slows and allows more blood to accumulate in the left ventricle, which allows the AV to open during the contraction of the heart. On the other hand, if the AV is opening every beat, the LVAD system controller 104 may increase the RPM of the LVAD 102 so that the flow through the LVAD 102 increases and allows less blood to accumulate in the left ventricle, which may require the AV to open less often and provide better blood flow to the body.

In addition to adjusting the RPM of the LVAD 102, the present systems and methods may also indicate the relative health of a patient's 101 heart. A physician may use this information to diagnose and/or make treatment decisions for the patient 101. For example, patients 101 may receive an LVAD 102 after a heart attack or heart surgery. Then, after some time of healing the LVAD 102 may be meant to be removed and the patient 101 may be considered recovered. However, it may be difficult to predict if and when a patient 101 will recover. In addition, it is difficult to know when the best time is to take out the LVAD 102. There may be a window of opportunity during which it is best to take out the LVAD 102 after the patient 101 is considered to be healed. If this window of opportunity is missed then the patient 101 may require a heart transplant or use of the LVAD 102 indefinitely rather than keeping their own heart, which may be more beneficial.

Therefore, in order to determine the relative strength of the heart, the magnitude of the projection of the electric current data onto the first eigenvector may indicate the contractility of the heart, i.e., how hard the native heart is contracting. This measurement could be plotted over time. The derivative of this plot reaching zero may signify that the maximum contractility has been reached, which would be the best time to take out the LVAD 102. In addition, the plot may reveal whether the heart contractility was increasing, signifying recovery.

Additionally, the electric current used by the LVAD 102 may be used to accurately indicate the rate of blood flow through the LVAD 102. Currently, some LVADs 102 are unable to accurately indicate the rate of blood flow. By integrating the area under the electric current curve and scaling the result, the LVAD system controller 104 may accurately indicate the rate of blood flow through the LVAD 102. This may assist physicians in treating patients 101 with LVADs 102.

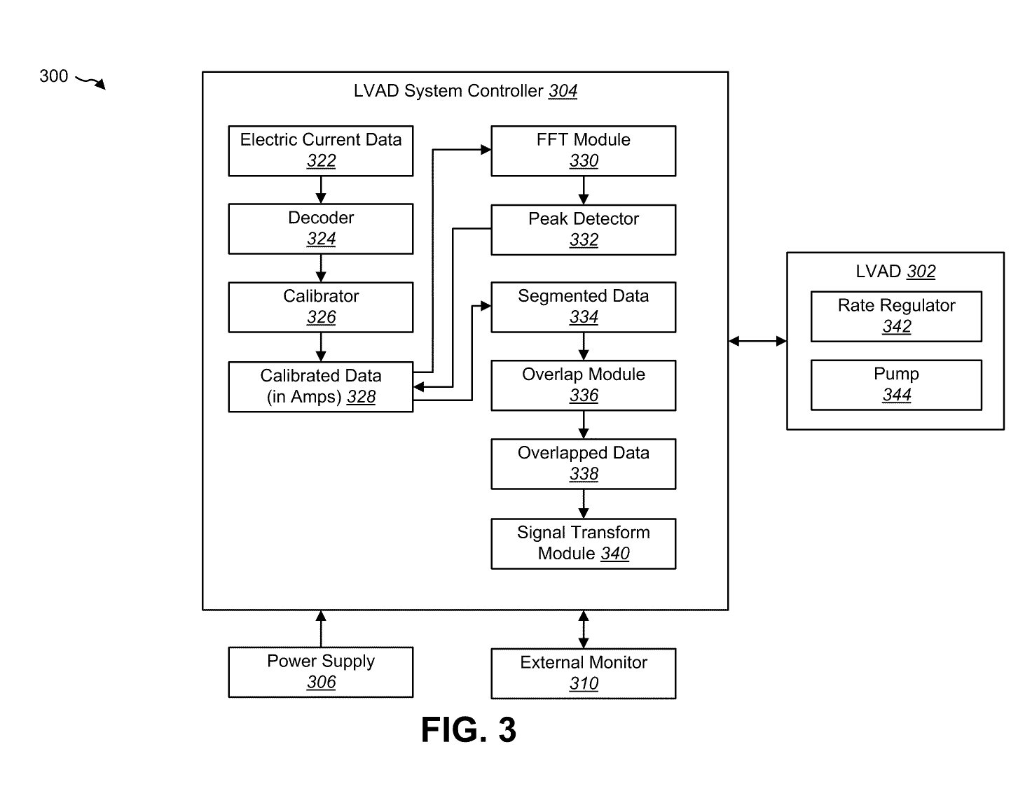

FIG. 3 is a block diagram illustrating a system 300 for determining whether an aortic valve is opening in an LVAD 302 patient 101. The system 300 may include an LVAD system controller 304, an LVAD 302, a power supply 306, and an external monitor 310. The LVAD system controller 304 may be configured to continuously monitor and regulate the RPM of the LVAD 302.

The LVAD 302 may receive electric current data 322 from the LVAD 302. The electric current data 322 may be data that indicates the electric current used by the LVAD 302 over a period of time, e.g., 10 seconds. The electric current data 322 may be in a proprietary format that is not well suited for signal processing. For example, electric current data 322 may not indicate actual data points. Therefore, a decoder 324 may decode the electric current data 322 into a more suitable format. However, the output of the decoder may indicate relative electric current, but not actual electric current used by the LVAD 302. In other words, the decoded data may not be in terms of Amperes. Therefore, a calibrator 326 may scale the decoded data into calibrated data 328 that is in units of Amperes. The calibrated data 328 may represent the electric current data 322 in a format more suitable for signal processing.

The LVAD system controller 304 may then filter the calibrated data 328 using an FFT module 330 and use a peak detector 332 to identify the systoles in the calibrated data 328. Systole may be indicated by valleys in the filtered output of the FFT module 330. In other words, the filtered calibrated data 328 may be sinusoid-like data representing the electric current usage of the LVAD 302 as a function of time. Each valley in this data may represent a contraction of the heart, i.e., a systole. The peak detector 332 may detect the time within the calibrated data 328 of the systoles. The calibrated data 328 may then be segmented to form segmented data 334 where each segment includes one systole. The segments in the segmented data 334 may then be transformed by an overlap module 336 to produce overlapped data 338. In other words, the overlapped data 338 may have the segments from the segmented data 334 overlapped with the systoles aligned at time 0. Using the overlapped data 338, a signal transform module 340 may determine whether an AV in a patient 101 is opening or continually closed. This determination may then be used to adjust the RPM of the LVAD 302. For example, a signal may be sent to a rate regulator 342 that alters the operation of a pump 344 in the LVAD 302. Alternatively, the rate regulator 342 may be in the LVAD system controller 304.

One purpose of overlapping cyclic data may be that when you analyze it the consistencies are more pronounced. Alternatively, the data may not be overlapped if the noise in the signal is relatively low. For example, FFT may be performed on the electric current data 322, keeping only certain frequencies, and overlapping the data may not be necessary to determine a physiological characteristic. Therefore, in one configuration, the segmenting and overlapping may not be performed. In other words, an alternative configuration may estimate peaks and valleys with previous known values, thus avoiding the overlapping.

Thus, in one configuration using PCA, it is possible to detect statistically significant changes when the AV is opening or continuously closed. This non-invasive analysis may help clinicians without echocardiograms determine an optimal LVAD 302 RPM to minimize AV stenosis and fusion, thrombosis, and potential neurological events. If this configuration were incorporated into the rotary non-pulsatile LVAD system controller 304, physiological feedback for an auto-regulating mode could be provided, which presently does not exist.

This AV opening detection algorithm may allow the LVAD system controller 304 to be programmed to automatically and continuously regulate the LVAD 302 RPMs. Continuous monitoring may decrease the patient's 101 risk for neurological events and AV stenosis or fusion.

At times, the AV may never open despite adjusting the RPM of the LVAD 302. For some patients 101, it may be dangerous to decrease the RPM and compromise blood flow to the point that the heart is able generate enough pressure to open the AV. Because some patients' 101 AVs will never open, despite decreasing the RPMs, an adjunct algorithm may be necessitated to prevent the LVAD system controller 304 from decreasing the RPM excessively low. For example, the LVAD system controller 304 may drop the RPM of the LVAD 302 down at a set interval, frequent enough to still prevent thrombosis formation and AV fusion.

Although the present systems and methods determine whether the AV is opening at a given RPM, there is no generally accepted ideal opening ratio. With this new current waveform analysis approach, the mechanical circulatory support field will be better equipped with a tool to determine what the ideal ratio is to optimize LVAD 302 therapy. Furthermore, the present systems and methods are adaptable for any ratio desired, e.g., AV opening once every 3 beats of the heart.

As before, the LVAD system controller 304 may receive power from a power supply 306, e.g., battery or AC power outlet. The LVAD system controller 304 may then supply power to the LVAD 302. If the signal transform module 340 determines that the RPM of the LVAD 302 needs adjusting, the power supplied to the LVAD 302 may also be adjusted. Also, an external monitor 310 may be used to perform some of the function of the LVAD system controller 304, and may also display various data related to the electric current usage of the LVAD 304.

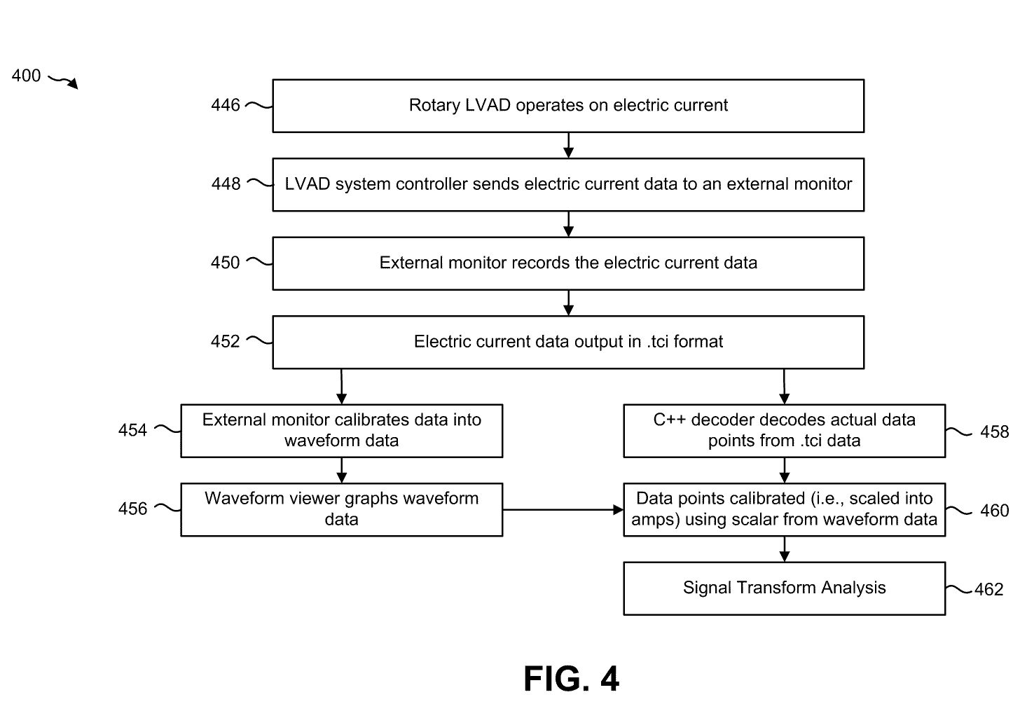

FIG. 4 is a flow diagram illustrating a method 400 for preparing data before determining whether an aortic valve is opening in an LVAD 302 patient 101. As discussed above, the electric current data 322 received by the LVAD system controller 304 may not be suitable for signal processing. Therefore, the electric current data 322 may need to be processed to produce a usable data set that is conducive to further signal processing to determine whether an AV is opening or not. The method 400 may be performed by one or all of the devices illustrated in FIG. 3, e.g., LVAD system controller 304, the LVAD 302, and the external monitor 310. In other words, the method 400 is only one configuration of possible methods for preparing data before determining whether an aortic valve is opening in an LVAD 102 patient 101. For example, in an alternative configuration, the LVAD 302 may send the electric current data 322 to the LVAD system controller 304 in a usable format, thus eliminating the need for calibrating the data into units of Amperes.

In the method 400, the rotary LVAD 302 may operate 446 using electric current. The LVAD system controller 304 may receive electric current data 322 and send 448 the electric current data 322 to an external monitor 310. The external monitor 310 may record 450, or store, the electric current data 322. The electric current data 322 may then be output 452 in a proprietary format to the LVAD system controller 304, e.g., Thermo Cardiosystems Incorporated (.tci) format. Alternatively, the LVAD system controller 304 may intercept the initial electric current data 322 from the LVAD 302. The external monitor 310 may calibrate 454 the proprietary data into waveform data and graph 456 the waveform data. A decoder 324 on the LVAD system controller 304 may decode 458 the proprietary data into actual data points. At this point, the data may indicate relative current usage in the LVAD 302, i.e., the data may not be in units of Amperes. The data points may then be calibrated 460 using a scalar derived from the waveform data, e.g., 0.00146. This may produce calibrated data 328 that is ready for further processing and signal transform analysis 462.

In one configuration, electric current waveforms for a HeartMate II LVAD may be recorded using Thoratec's external display modules. All electric current waveform files, (i.e., electric current data 322), may be saved by the equipment in *.tci format. In order to extract the data from *.tci files, a Minimalist GNU for Windows (MinGW), Minimal System (MSYS) console, and a C++ decoder may be used to properly inter-digitate the data into a single data vector. To prepare the raw electric current data 322, the MSYS console may first point the desired *.tci file into the C++ code to output a *.dat file, which may then be loaded into a numerical computing environment, (e.g., MatLab), and analyzed. Alternatively, the electric current data 322 may be extracted using only MatLab and not MinGW or MSYS. The analysis may begin by calibrating the unscaled *.dat file values, which may be proportional to electric current, into values with units of Amperes by a multiplication factor of 0.00146. In other words, the multiplication factor may be used to produce calibrated data 328 that is in Amperes. This Ampere calibration factor may be determined by comparison of known pre-determined Thoratec (the manufacturer of the HeartMate II LVAD) Ampere values calculated from Thoratec's Current Waveform Viewer application. The output of the Current Waveform Viewer application may be unfiltered current data in Amperes, i.e., calibrated data 328. This current data may need to be intercepted at an earlier stage in order to collect actual values for each data point, thus the need to calibrate prior to analysis.

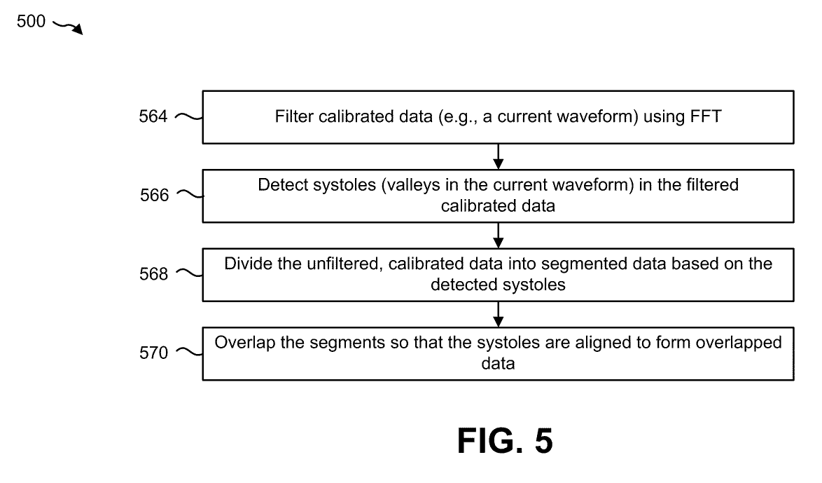

FIG. 5 is a flow diagram illustrating a method 500 for filtering and organizing data before determining whether an aortic valve is opening in an LVAD 302 patient 101. In other words, steps 564-570 may be performed in place of step 216 of the method 200 illustrated in FIG. 2. The method 500 may be performed by an LVAD system controller 304. The electric current data may be filtered 564 using a low-pass filter FFT until a single waveform is captured. The calibrated data 328 may have been produced by the method 400 of FIG. 4.

Subsequently, the derivative of the filtered current waveform (i.e., the filtered calibrated data 328) may be analyzed to determine the beginning of a heart beat by its derivative being negative in value and subsequently positive. In addition, a heart contraction may be detected when a slope of 1.5 e-4 Amps/millisecond is sustained in any given 225 millisecond current interval for 125 milliseconds. These values may be determined by maximizing the accepted heart contractions of the current waveform while rejecting the partial heart contractions recorded at the beginning and the end of the recording interval. If the first heart contraction data point is skewed due to its position in the recording cycle, it may be disregarded to avoid calculation errors. Once the beginning of the full recorded heart contractions was found, the following 550 ms of current data was extracted. In other words, the LVAD system controller 304 may detect 566 systoles (i.e., valleys) in the filtered calibrated data 328, divide 568 the unfiltered, calibrated data 328 into segmented data 334 based on the detected systoles, and overlap 570 the segments so that the systoles are aligned to form overlapped data 338, e.g., in matrix form. The overlapped data 338 may then be used for further signal processing, such as PCA, to determine whether an AV is opening in an LVAD 302 patient 101. For example, each row in an overlapped data 338 matrix may represent the first 550 ms or 600 ms of a particular heart beat. Therefore, each column in the overlapped data 338 may represent each electrical ms sample in amps. A covariance matrix may be calculated from this data, and eigenvalues and eigenvectors may be calculated from the covariance matrix.

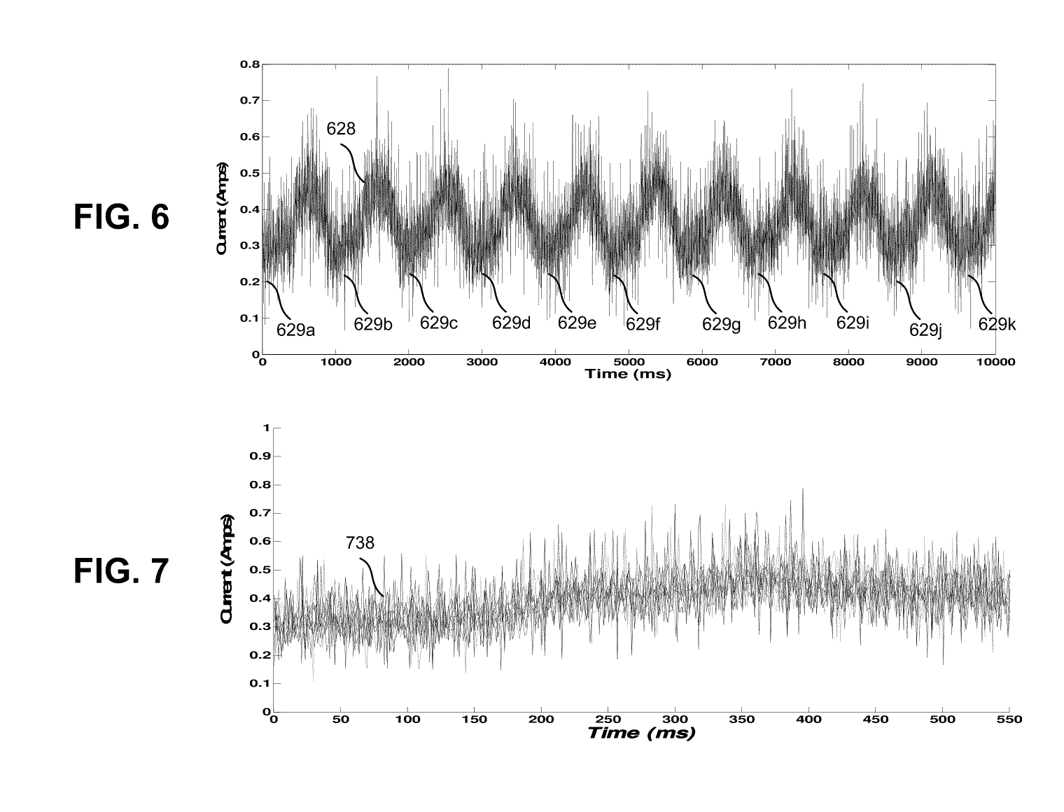

FIG. 6 is a waveform illustrating calibrated data 628. The calibrated data 628 may be in a usable format for further signal processing, i.e., the calibrated data 628 may be the electric current data 322 from the LVAD 302 after decoding and calibration. In one configuration, the data 628 may represent the original ten second HeartMate II current waveform after calibration in MatLab. Rises in the electric current may depict systolic contraction, whereas downward slopes may depict diastole. The valleys in the calibrated data 628 may indicate systoles 629. In other words, the waveform may include eleven systoles 629a-k. The data sampling frequency for the calibrated data 628 is 1.00 ms⁻¹, although other sampling frequencies may be used. Although the LVAD 302 is non-pulsatile and a continuous flow rotor device, pulsatility may be introduced into the system because of the native heart's cyclic contraction.

FIG. 7 is a waveform illustrating overlapped data 738. The overlapped data 738 may be the calibrated data 628 that has been filtered, segmented at systoles 629, and overlapped, i.e., one ten second current waveform split into the systolic intervals of interest where the systoles 629 are aligned at time 0. In other words, the data 738 may represent electric current during the systolic intervals of interest over a 10 second period where each systolic contraction 629 begins at time zero. These systolic intervals, from the initialization of the heart contraction to 550 ms post-initialization, may be stored into a master matrix for all calibrated current *.dat waveforms recorded, i.e., overlapped data 738.

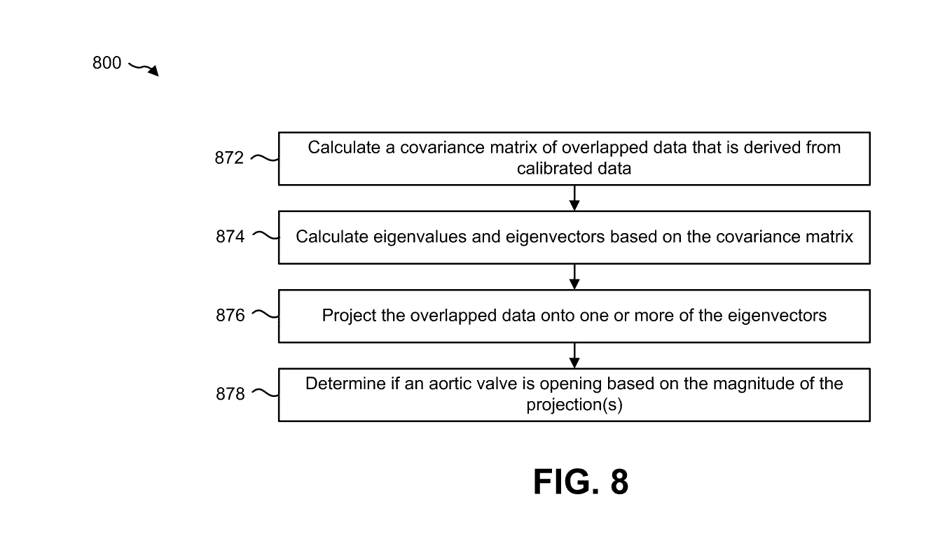

FIG. 8 is a flow diagram illustrating a method 800 for Principal Component Analysis (PCA). Once the overlapped data 738 is produced, the LVAD system controller 304 may perform additional signal transformation. While PCA is illustrated in the method 800, any suitable signal processing technique may be used. The method 800 may be performed by the LVAD system controller 304.

The LVAD system controller 304 may calculate 872 a covariance matrix of the overlapped data 738 that is derived from calibrated data 628. The components of the symmetric covariance matrix may be calculated using equation (1):

cov(Yᵢ,Yⱼ)=E[(Yᵢ−μₓⱼ)] (1)

where the i and j indices run from 1 to the number of observations in the dataset (n=551), E is the mathematical expectation and μᵢ=E(Y_{i/j}), and Y=current in amps. The right eigenvalues and eigenvectors of the shown covariance matrix (M) may then be calculated 874 using equation (2):

(M−λ_{R}I)X_{R}=0 (2)

where λ_{R} represents the right eigenvalues, X_{R} represents the right eigenvectors, and I represents the identity matrix. Once the eigenvectors are calculated 874, the original electrical signal, (i.e., the calibrated data 628), may be projected 876 onto each of the eigenvectors. In one configuration, 10 eigenvectors may be used, although as many eigenvectors as the covariance matrix is long may be analyzed.

As mentioned above, eigenvectors may indicate shared signal behavior. The eigenvalues associated with the eigenvectors may indicate the relative importance of each eigenvectors' contribution to the original data, i.e., the overlapped data 738. Therefore, the eigenvalues may be used to rank the consistency or contributing power to the overall signal. A particular eigenvector's percentage of contribution to the overall signal may be calculated by dividing the eigenvector' s associated eigenvalue by the sum of all eigenvalues, multiplied by 100.

The raw data projected onto each of the eigenvectors may be analyzed using Student's T-tests to determine trends of electric current and AV movement as the RPM of the rotary LVAD 302 (e.g., HeartMate II) is adjusted. In other words, the LVAD system controller 304 may determine 878 if an AV is opening based on the magnitude of the projections onto one or more of the eigenvectors. For example, the projection may include performing a running dot product of the overlapped data 738 with one or more eigenvectors. An eigenvector of length n (which is approximately 18 times smaller than a recorded sample due to the overlapping of the data to calculate the eigenvector) may be projected onto the first n elements of the recorded sample. This single dot product value may be plotted. The eigenvector may then projected onto elements 2 to n+1 and the dot product value may then be plotted next to the first. Again, the eigenvector may be projected onto elements 3 to n+2 and plotted. This process may be repeated until the eigenvector cannot be fully projected onto the recorded data. This auto-correlation method may emphasize similarities of the eigenvector in the recorded sample, thus revealing shared signal behavior, if it exists. Actual AV opening ratios may be determined by recording the current waveforms at the time of an echocardiogram procedure using motion mode. While the present systems and methods are described using the overlapped data 738 in the signal transform module 340, the calibrated data 628 may also be used by the signal transform module 340 to determine whether an AV is opening. In other words, eigenvector(s) may be projected onto the calibrated data 628 instead of or in addition to the calibrated overlapped data 738.

In one configuration, the first eigenvector correlates with the AV opening. Thus, the LVAD system controller 304 may use the electrical signal, (i.e., overlapped data 738), projected onto the first eigenvector to determine 878 the AV opening ratio. Therefore, the LVAD system controller 304 may only project 876 the overlapped data 738 onto the first eigenvector since it is the most contributing sub-signal. Then, from this projection, it may be determined 878 if an AV is opening.

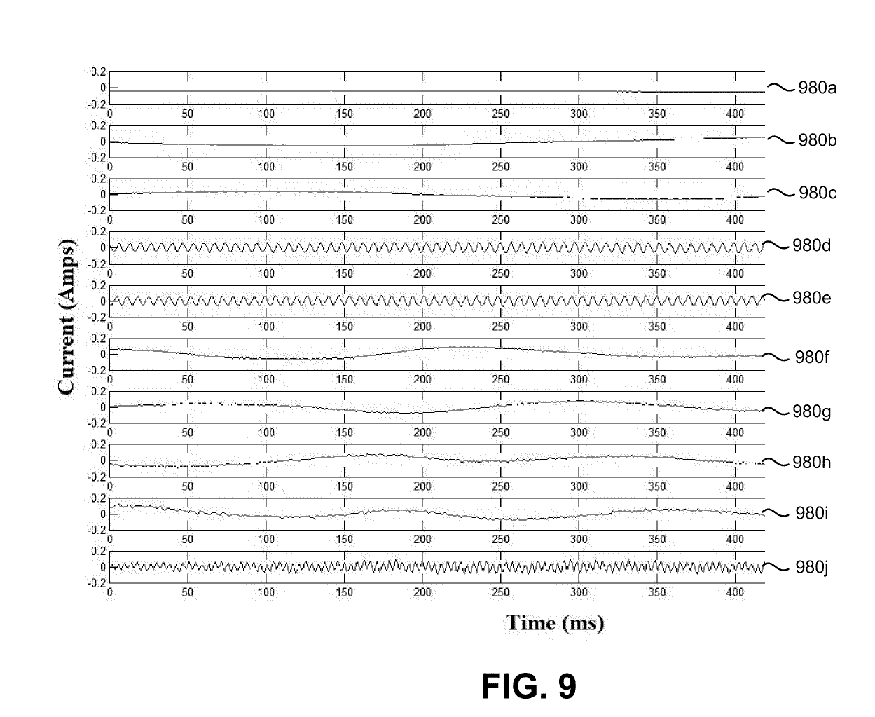

FIG. 9 is a waveform illustrating eigenvectors 980. In other words, examples of eigenvectors 980 that satisfy Equation (2) are illustrated in FIG. 9. Waveforms 980a-j correspond to eigenvectors 1-10, respectively. The eigenvectors 980 may represent the different components of the overlapped data 738. In one configuration, overlapped data 738 may be projected on one or more of the eigenvectors 980 (e.g., the first eigenvector 980a), in order to determine whether an AV is opening.

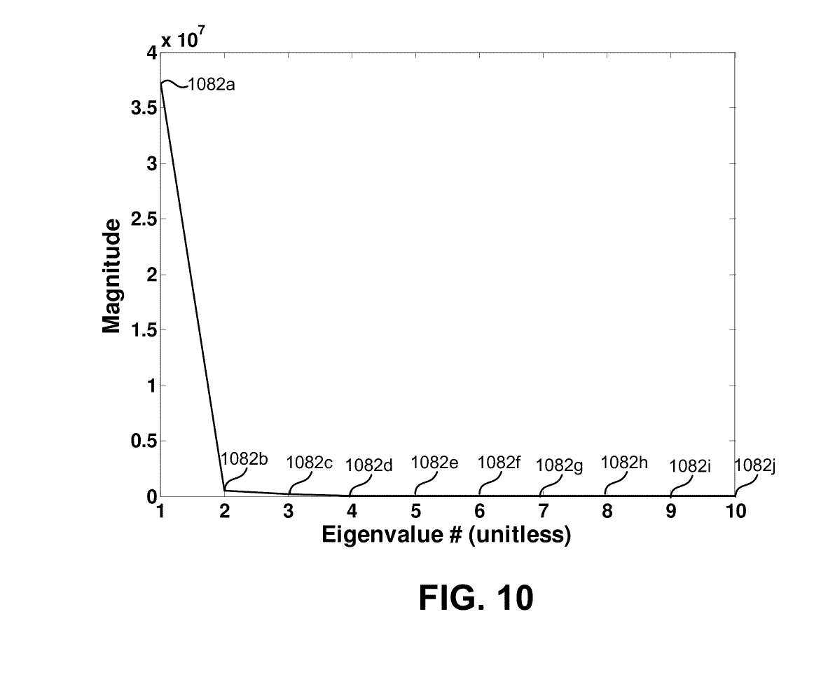

FIG. 10 is a waveform illustrating eigenvalues 1082. In other words, examples of eigenvalues 1082 that satisfy Equation (2) are illustrated in FIG. 10. Values 1082a-j correspond to eigenvalues 1-10, respectively. As discussed earlier, an eigenvalue 1082 may represent the relative contribution of its associated eigenvector 980 to the entire signal, i.e., the calibrated data 328. Thus, the first eigenvalue 1082a may indicate that the first eigenvector 980a represents a much larger contribution to the entire signal, (i.e., the calibrated data 328), than any of the other eigenvectors 980b-j because the first eigenvalue 1082a is much larger than the other eigenvalues 1082b-j.

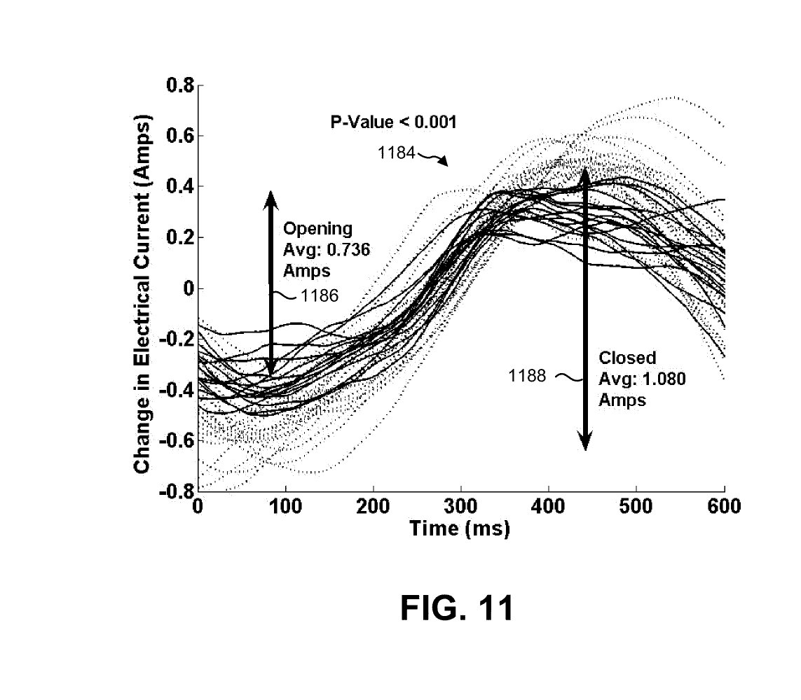

FIG. 11 is a waveform illustrating the projections 1184 of data onto the first eigenvector 980a. In other words, FIG. 11 illustrates the projection 1184 of the original current signal onto the first eigenvector 980a when the AV is opening and when it is continuously closed. The black solid waveforms may represent the projections 1184 when the AV is opening while the dotted-line waveforms may represent the projections 1184 when the AV is continuously closed. The magnitude 1186 of the signal is 0.736 amps when the AV is opening and the magnitude 1188 of the signal is 1.080 amps when the AV is closed. Thus, the magnitude of the current used by the LVAD 302 increases significantly once the AV is closed. When the AV is opening, less blood is traversing the LVAD 302 compared to when the AV is closed. Once the valve is closed, the LVAD 302 receives more blood and the current increases. Although peak to peak magnitude is illustrated, any method of comparing relative amplitudes may be used, e.g., peak amplitude, root mean square (RMS), etc. The low P-Value indicates a significant result when comparing the magnitude 1186 when the AV is opening and the magnitude 1188 when the AV is closed.

In other words, a vector dot product between the dominant eigenvector, (e.g., 600 data points long), and the first 600 ms of the raw electrical waveforms (in amps) may be used. Further dot products may be performed with the dominant (1st) eigenvector with data points 2-601, and then 3-602, etc. until 9401-10000 ms are completed and the dot product is no longer possible for lack of data points beyond 10000 ms. In other words, the term “projection” as used herein may refer to a running average or a running dot product. After this, each heart beat (first 600 ms of each heart beat) of the projection may be time aligned at t=0.

Magnitudes may be used to determine if the AV is opening, however, the presence or absence of a sub-wave for a given eigenvector projection may also be used as an indicator for AV opening/closing. Any characteristic in the waveform, whether it be frequency, magnitude, etc., may be used to identify significant differences indicating when the aortic valve is opening or continuously closed.

The present systems and methods may be adapted for individual patients 101. For example, Table 1 illustrates data for six patients that were analyzed with echocardiography comparisons. The table shows the average magnitude of the electric current signal when the AV is opening versus closed. Patients 101 included were those with AVs that were always closed or always opened with adjustments of the RPM of the LVAD 302. In all cases, the electric current magnitude change when the AV stopped opening was different. Four of the 6 patients' 101 current increase was statistically significant, indicated by the p-values <0.05 (α=0.05). The 2 patients 101 whose increase was not statistically significant (patient 4 and patient 6 in Table 1) had mild AV regurgitation. The present systems and methods may be adapted for AV regurgitation.

TABLE 1

| | Avg. | Avg. | |
|---|---|---|---|
| | Opening | Closed | |
| Patient | Mag. | Mag. | P-value |
| 1 | 0.736 | 1.080 | <0.001 |
| 2 | 1.849 | 2.366 | 0.014 |
| 3 | 1.224 | 1.711 | 0.003 |
| 4 | 0.883 | 1.079 | 0.204 |
| 5 | 1.760 | 1.894 | 0.014 |
| 6 | 0.616 | 0.699 | 0.242 |

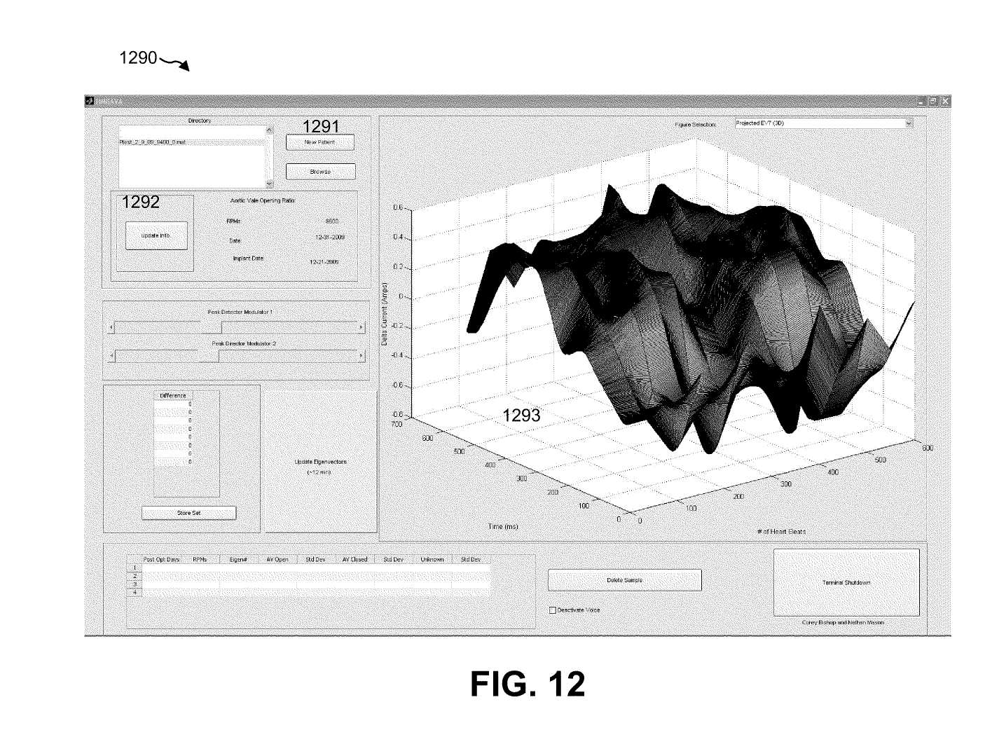

FIG. 12 is a screenshot 1290 illustrating one possible configuration of an LVAD aortic valve opening analyzer clinical application. Such an application may include a graphical user interface (GUI) with various functionality. For example, the interface may include a patient selection section 1291, a specification input section 1292, a graphical data viewing section 1293, etc. The GUI may use buttons, dropdown menus, slider bars, or any suitable input or display mechanism.

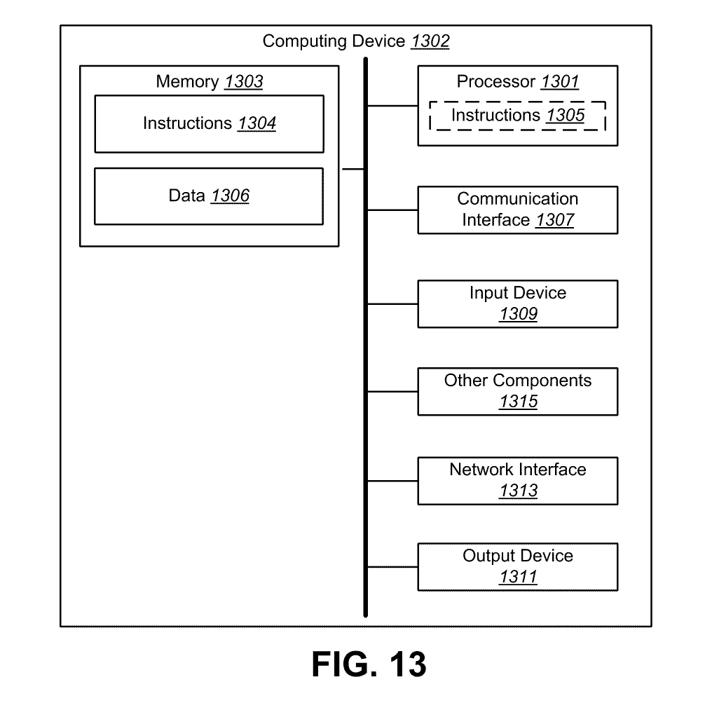

FIG. 13 is a block diagram illustrating various components that may be utilized in a computing device 1302 in a system 100 for determining whether an AV is opening in LVAD 102 patients 101. For example, the computing device 1302 may be used to implement an LVAD 102, an LVAD system controller 104, an external monitor 110, or a computer 112. Although only one computing device 1302 is shown, the configurations herein may be implemented in a distributed system using many computing devices.

The computing device 1302 is shown with a processor 1301 and memory 1303. The processor 1301 may control the operation of the computing device 1302 and may be embodied as a microprocessor, a microcontroller, a digital signal processor (DSP) or other device known in the art. The processor 1301 typically performs logical and arithmetic operations based on program instructions stored within the memory 1303. The instructions 1304 in the memory 1303 may be executable to implement the methods described herein.

The computing device 1302 may also include one or more communication interfaces 1307 and/or network interfaces 1313 for communicating with other electronic devices. The communication interface(s) 1307 and the network interface(s) 1313 may be based on wired communication technology, wireless communication technology, or both.

The computing device 1302 may also include one or more input devices 1309 and one or more output devices 1311. The input devices 1309 and output devices 1311 may facilitate user input. Other components 1315 may also be provided as part of the computing device 1302.

Data 1306 and instructions 1304 may be stored in the memory 1303. The processor 1301 may load and execute instructions 1305 from the instructions 1304 in memory 1303 to implement various functions. Executing the instructions 1304 may involve the use of the data 1306 that is stored in the memory 1303. The instructions 1304 are executable to implement one or more of the processes or configurations shown herein, and the data 1306 may include one or more of the various pieces of data described herein.

The memory 1303 may be any electronic component capable of storing electronic information. The memory 1303 may be embodied as random access memory (RAM), read only memory (ROM), magnetic disk storage media, optical storage media, flash memory devices in RAM, on-board memory included with the processor, EPROM memory, EEPROM memory, an ASIC (Application Specific Integrated Circuit), registers, and so forth, including combinations thereof.

The phrase “based on” does not mean “based only on,” unless expressly specified otherwise. In other words, the phrase “based on” describes both “based only on” and “based at least on.”

The term “processor” should be interpreted broadly to encompass a general purpose processor, a central processing unit (CPU), a microprocessor, a digital signal processor (DSP), a controller, a microcontroller, a state machine, and so forth. Under some circumstances, a “processor” may refer to an application specific integrated circuit (ASIC), a programmable logic device (PLD), a field programmable gate array (FPGA), etc. The term “processor” may refer to a combination of processing devices, e.g., a combination of a DSP and a microprocessor, a plurality of microprocessors, one or more microprocessors in conjunction with a DSP core, or any other such configuration.

The term “memory” should be interpreted broadly to encompass any electronic component capable of storing electronic information. The term memory may refer to various types of processor-readable media such as random access memory (RAM), read-only memory (ROM), non-volatile random access memory (NVRAM), programmable read-only memory (PROM), erasable programmable read only memory (EPROM), electrically erasable PROM (EEPROM), flash memory, magnetic or optical data storage, registers, etc. Memory is said to be in electronic communication with a processor if the processor can read information from and/or write information to the memory. Memory may be integral to a processor and still be said to be in electronic communication with the processor.

The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may comprise a single computer-readable statement or many computer-readable statements.

The term “computer-readable medium” refers to any available medium that can be accessed by a computer. By way of example, and not limitation, a computer-readable medium may comprise RAM, ROM, EEPROM, CD-ROM or other optical disk storage, magnetic disk storage or other magnetic storage devices, or any other medium that can be used to store desired program code in the form of instructions or data structures and that can be accessed by a computer. Disk and disc, as used herein, includes compact disc (CD), laser disc, optical disc, digital versatile disc (DVD), floppy disk and Blu-ray® disc where disks usually reproduce data magnetically, while discs reproduce data optically with lasers.

Software or instructions may be transmitted over a transmission medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of transmission medium.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

It is to be understood that the claims are not limited to the precise configuration and components illustrated above. Various modifications, changes and variations may be made in the arrangement, operation and details of the systems, methods, and apparatus described herein without departing from the scope of the claims.

What is claimed is:

1. A method for determining a physiological characteristic of a patient on which a medical device is applied, comprising:
obtaining electric current data from the medical device;
filtering the electric current data to produce filtered data; and
determining the physiological characteristic based on the filtered data.

2. The method of claim 1, wherein the medical device is a left ventricular assist device (LVAD).

3. The method of claim 2, wherein the physiological characteristic is whether an aortic valve in the patient is opening.

4. The method of claim 1, wherein the determining comprises using Principal Component Analysis.

5. The method of claim 4, wherein the determining further comprises:
determining a covariance matrix based on the filtered data;
calculating eigenvalues and eigenvectors based on the covariance matrix;
projecting the eigenvectors onto the filtered data; and
determining the physiological characteristic based on a characteristic of the projection.

6. The method of claim 3, further comprising adjusting the rotations per minute (RPM) of a pump in the left ventricular assist device (LVAD) based on the determination.

7. The method of claim 1, wherein the filtering comprises using Fast Fourier Transform (FFT).

8. The method of claim 2, further comprising determining a rate of blood flow through the left ventricular assist device (LVAD) by integrating an area under a curve defined by the electric current data.

9. The method of claim 1, further comprising determining contractility of the patient's heart using a magnitude of the electric current data that is proportional to contractility when volume status is normalized.

10. The method of claim 9, further comprising determining whether to remove the left ventricular assist device (LVAD) from the patient based on the contractility determination.

11. The method of claim 1, wherein the physiological characteristic is a blood pressure of the patient.

12. An apparatus for determining a physiological characteristic of a patient on which a medical device is applied, the apparatus comprising:
a processor;
memory in electronic communication with the processor; and
instructions stored in the memory, the instructions being executable to:
obtain electric current data from the medical device;
filter the electric current data to produce filtered data; and
determine the physiological characteristic based on the filtered data.

13. The apparatus of claim 12, wherein the medical device is a left ventricular assist device (LVAD).

14. The apparatus of claim 13, wherein the physiological characteristic is whether an aortic valve in the patient is opening.

15. The apparatus of claim 12, wherein the instructions executable to determine comprise instructions executable to use Principal Component Analysis.

16. The apparatus of claim 15, wherein the instructions executable to determine further comprise instructions executable to:
determine a covariance matrix based on the filtered data;
calculate eigenvalues and eigenvectors based on the covariance matrix;
project the eigenvectors onto the filtered data; and
determine the physiological characteristic based on a characteristic of the projection.

17. The apparatus of claim 14, further comprising instructions executable to adjust the rotations per minute (RPM) of a pump in the left ventricular assist device (LVAD) based on the determination.

18. The apparatus of claim 13, further comprising instructions executable to determine a rate of blood flow through the left ventricular assist device (LVAD) by integrating an area under a curve defined by the electric current data.

19. The apparatus of claim 12, further comprising instructions executable to determine contractility of the patient's heart using a magnitude of the electric current data that is proportional to contractility when volume status is normalized.

20. A computer-readable medium comprising executable instructions for:
obtaining electric current data from a medical device applied to a patient;
filtering the electric current data to produce filtered data; and
determining a physiological characteristic of the patient based on the filtered data.
